(19) United States
(12) Patent Application Publication
(10) Pub. No.: US 2024/0374317 A1
(43) Pub. Date: Nov. 14, 2024

(54) PHYSIOLOGICALLY SYNCHRONIZED SURGICAL PROCEDURES

(71) Applicant: ClearPoint Neuro, Inc., Solana Beach, CA (US)

(72) Inventors: Nathan Williams, San Diego, CA (US); Christian Osswald, Roselle, IL (US)

(21) Appl. No.: 18/658,295

(22) Filed: May 8, 2024

Related U.S. Application Data
(60) Provisional application No. 63/500,778, filed on May 8, 2023.

(51) Int. Cl. A61B 34/20

(52) U.S. Cl. CPC A61B 34/20; A61B 2034/2074

(57) ABSTRACT

Systems and methods are configured to synchronize a medical device intervention wherein the anatomy moves or changes shape relative to the medical device. A medical device is positioned relative to anatomy. Data is then obtained regarding a position of the anatomy. The data is used to synchronize a position of the medical device relative to the anatomy such as to maintain a relative position between the medical device and the anatomy such as a specific portion of the anatomy.

### CROSS REFERENCE TO RELATED APPLICATION

This application claims the benefit of priority to U.S. Provisional Patent Application No. 63/500,778, entitled “PHYSIOLOGICALLY SYNCHRONIZED SURGICAL PROCEDURES”, filed on May 8, 2023, which is hereby incorporated by reference herein in its entirety.

### BACKGROUND

In various medical intervention procedures, physical changes or movements in anatomy (e.g. pulsing of the anatomy with heart beats) may change the position of a medical device relative to the anatomy as the anatomy moves. This can cause a variety of negative issues, such as partial (or improper) insertion of the medical device into anatomy or the device being improperly positioned relative to the target anatomy.

Pursuant to a medical device intervention with anatomy, the medical device can make direct contact with anatomy such that medical device passively moves with the anatomy as the anatomy's movement causes corresponding movement of the medial device. For example, many tasks are performed manually such that a physician moves the medical device as the anatomy moves. Or the physician attempts to time a movement action to correspond with a specific physiological state. For example, a brain-computer interface (BCI) implant procedures involves a surgeon manually positioning the implant relative to the brain and then pushing a trigger button to insert the implant into the brain while the brain is pulsing. The time delay and inconsistent nature of brain pulsations can often result in incomplete implantation or imprecise determination of anatomical state.

In another example, rather than trying to compensate for the change in physiological state during the procedure, the physician may simply inhibit the change in physiological state (such as anatomical movements) by fixing the anatomy (e.g. restraining the patient) or by arresting a physiological function (e.g. temporarily stopping respiration or heart beating.)

### SUMMARY

Disclosed are systems and methods configured to adjust a position of a medical device relative to anatomy to compensate for movement of the anatomy relative to the medical device during an intervention procedure.

In one aspect, there is disclosed a method of synchronizing a medical device intervention, comprising: positioning a medical device relative to anatomy; obtaining data regarding a position of the anatomy; and using the data to synchronize a position of the medical device relative to the anatomy. In another aspect, at least one memory including computer program code are configured to, with at least one data processor, cause a system to implement the method.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

FIGS. 1A-1C schematically show sequential, relative movement of a medical device probe and adjacent anatomy.

FIGS. 2A-2C schematically show sequential, relative movement of a medical device probe and anatomy.

FIGS. 3A-3C schematically show a properly timed insertion of a medical device probe relative to anatomy wherein implantation of the medical device probe matches the peak of a pulsation of the anatomy.

FIGS. 4A-4C schematically show a partial insertion of a medical device probe relative to anatomy due to the anatomy being, for example, at the bottom of a pulsation while the device is being inserted thereby resulting in an incomplete insertion of the medical device probe into the anatomy.

FIG. 5 shows a schematic representation of a system configure to maintain relative position between a medical device and anatomy.

FIG. 6 depicts a block diagram illustrating an example of a computing control system consistent with implementations of the current subject matter.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed are systems and methods configured to adjust, align and/or compensate for relative movement between a medical device and adjacent anatomy during a medical procedure. The relative movement may be the result of natural, physiological changes to the anatomy. A medical device may be held or otherwise positioned adjacent to, near, in contact with, or inserted into the anatomy for a variety of reasons. In a non-limiting example, a medical device may be inserted into the brain for diagnostic or therapeutic purposes. It can be critical that the medical device be positioned in a precise position relative to a target anatomy during a procedure. However, the anatomy can move relative to the medical device or other landmark for a variety of reasons. In a non-limiting example, the cortical surface of the brain ‘pulses’ or deflects up and down as the heart beats or as the patient breathes. If the medical device is positioned such that it is adjacent to the cortical surface, then the relative positions of the medical device and cortical surface can change as the heart beats or the patient breathes. Such a change in relative positions can be undesirable in certain situations.

It can be thus desirable to account for these physiological movements so that the medical device (or an implant or other element attached to the medical device) can be properly operated in accordance with the current state of the anatomy. The physiological movements can be accounted for in at least two different manners, including:
(1) Maintaining the position of a medical device relative to a target anatomy even as the target anatomy moves; and/or
(2) Performing a function of the medical device, such as an intervention on the anatomy, while the anatomy is in a specific, known anatomical state or position.

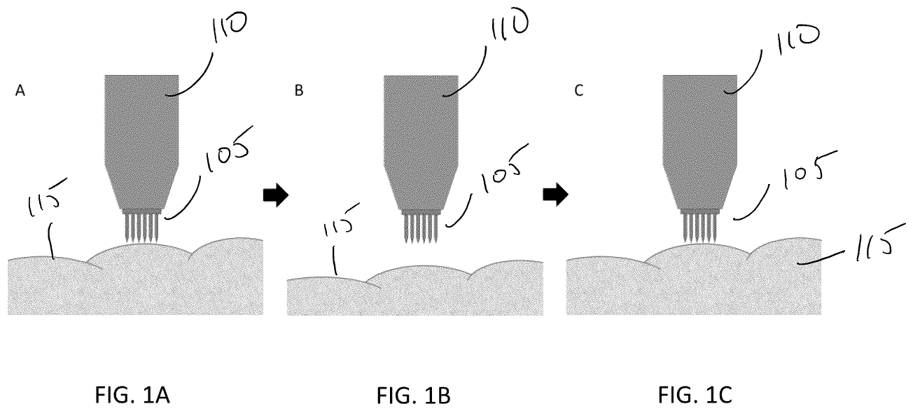

FIGS. 1A-1C schematically illustrate an example of the cortical surface of the brain pulsing, or deflecting up and down, as the heart beats or as the patient breathes. If the medical device is positioned such that it is adjacent to the cortical surface, then the relative positions of the medical device and cortical surface can potentially change as the heart beats or the patient breathes.

Each of FIGS. 1A-1C presents a sequential moment in time as relative movement occurs between the anatomy and the medical device. FIG. 1A shows a medical device comprising a probe 105 and a fixture 110 attached to a distal end of the probe 105. The probe 105 is held in a stationary position by the fixture 110. The probe 105 is making contact with, or may otherwise be positioned relative to, the anatomy comprising cortical surface 115 of the brain while the brain pulses. FIG. 1B shows the cortical surface 115 of the brain moving away from or ‘down’ (relative to the probe 105 and the fixture 110) between pulses such that the probe 105 no longer is in contact with the cortical surface 115. FIG. 1C shows that the probe 105 is again in contact with the cortical surface 115 due to the cortical surface moving toward or upward relative to the probe 105 during the next brain pulse. It should be appreciated that the medical device does not necessarily have to be maintained in direct contact with the anatomy. The medical device can be maintained or otherwise synchronized to be in a position, such as a fixed position, relative to the anatomy as the anatomy moves or change shape.

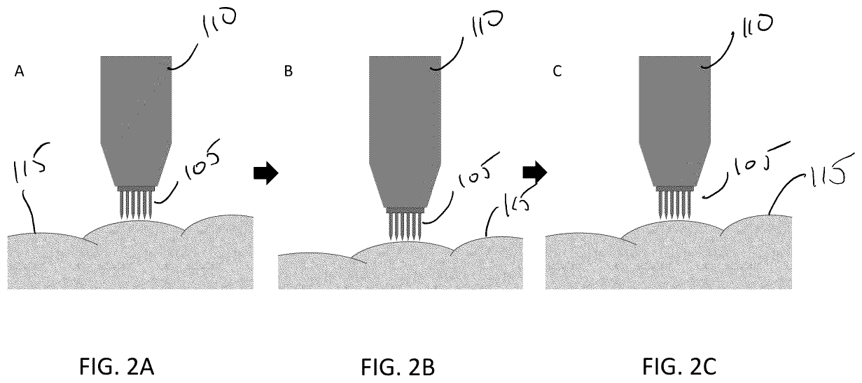

FIGS. 2A-2C illustrates an example of the systems and methods described herein wherein such system and methods are configured to maintain a position of a medical device relative to a target anatomy even as the target anatomy moves. During such anatomical movement, the medical device is caused to move in synchronization with the anatomy to maintain a relatively fixed position between the anatomy and a portion of the medical device, such as a distal end of the medical device and/or a probe of the medical device.

FIG. 2A shows the probe 105 and the fixture 110 with the probe 105 held in a stationary position by the fixture 110. That is, a distal tip of the probe 105 is in a position relative to a surface of the anatomy. As shown in FIG. 2B, the cortical surface 115 begins to moves down (or away from the probe 105) as a result of the brain pulsing. Pursuant to the methods described herein, the fixture 110 and the attached probe 105 also move down, such as by a corresponding amount of distance that the cortical surface 115 moved down. Such movement is in synchronization with the anatomical movement. In this manner, the relative positions between the probe 105 and the cortical surface 115 remains fixed or unchanged as the cortical surface 115 moves. In FIG. 1C, the cortical surface 115 has moved upward while probe 105 and fixture 110 also moves upward corresponding to an amount of cortical surface movement. In this way the probe 105 is maintained in a fixed position (such as in contact) relative to the cortical surface 115 even as the brain moves over time.

It may be desirable to use the medical device to perform a function on the anatomy (such as a medical intervention) at a specific moment while the anatomy is in a specific, known state. For example, a medical device comprising fixture 110 may move to push a probe m105 into the cortical surface 115 at a specific moment while the anatomy is in a specific, known state. It may be beneficial for this insertion to take place while the brain is pulsing and/or begin the insertion while the probe is already in contact with the cortical surface.

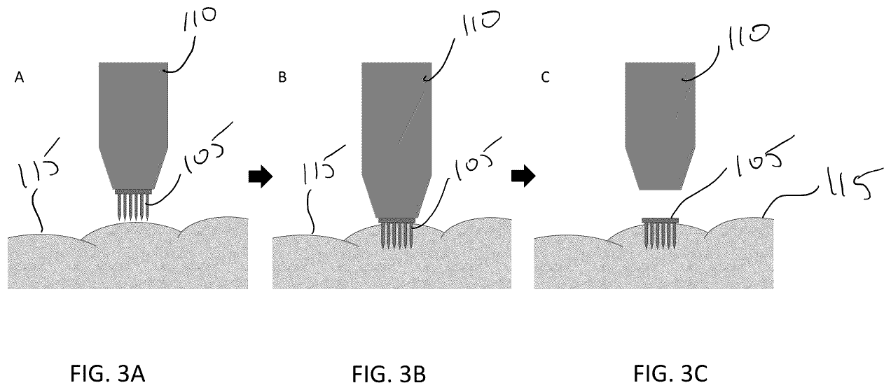

FIGS. 3A-3B illustrates an example of using the medical device to perform a function on the anatomy (such as a medical intervention) at a specific moment while the anatomy is in a specific, known state. FIG. 3A shows a medical device comprising a probe 105 and a fixture 110 with the probe 105 held in a stationary position by the fixture 110. FIG. 3B shows that the fixture 110 and the attached probe 105 have moved downward to insert at least a portion of the probe 105 into the brain while the brain is in the position corresponding to the same position as FIG. 3A. FIG. 3C shows the fixture 110 raised to its original position (corresponding to the position of FIG. 3A). The probe 105 is at least partially inserted into the brain. The process of FIGS. 3A-3C occurs very quickly so there is minimum change to the position of the brain during insertion of the probe 105.

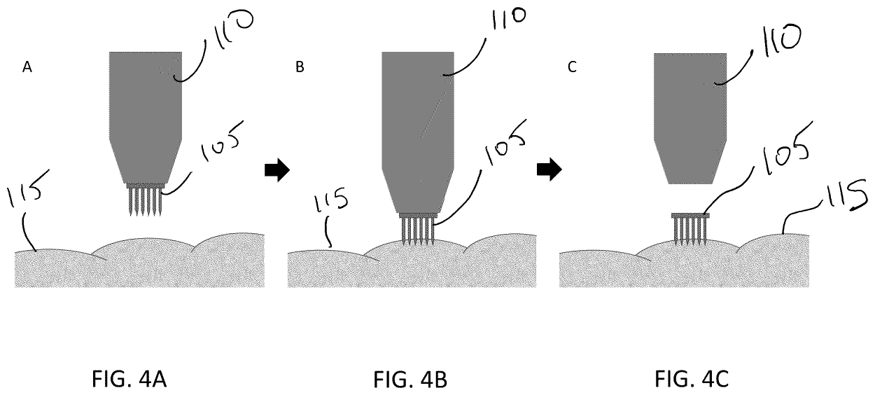

In contrast to FIGS. 3A-3C, FIGS. 4A-4C shows the probe 105 being inserted into the brain while the brain is between pulses. FIG. 4A shows the probe 105 distanced from and not in contact with the cortical surface 115. FIG. 4B shows that the fixture 110 has moved downward toward the brain to insert the probe 115 at least partially into the brain. The fixture 110 has moved the same distance as in FIG. 3A to FIGS. 3B, but in this case the probe 105 is not fully inserted into the brain because the cortical surface was farther away from the probe when the insertion occurred due to the pulsation movement of the brain. FIG. 4C shows the fixture 110 raised to its original position and the probe 105 at least partially inserted into the brain.

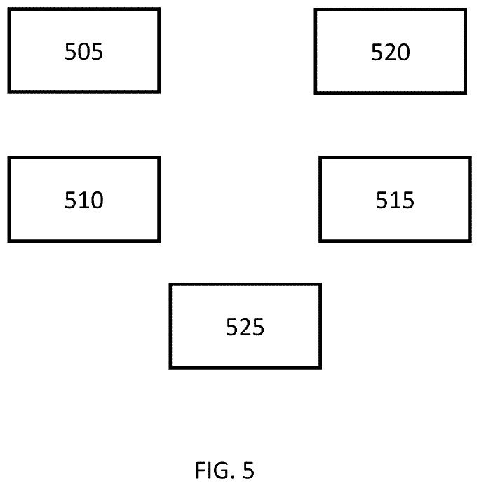

FIG. 5 shows a schematic representation of the system including the medical device 510 and anatomy 515. The medical device can vary in configuration and can be, for example, a catheter, a probe, a cannula, etc. The medical device 510 and the anatomy 515 are physically and/or communicatively coupled to one another and to other device, such as a sensor 505, a position measuring device 520 or other devices such as physiological measurements or monitor(s) 525.

The system is configured to obtain and act on at least some data or knowledge of the anatomical state at a given point in time. This can be accomplished in a variety of ways. For example, the system can include at least one sensor 505 coupled to the medical device 510 and to the anatomy wherein the sensor provides data relevant to a distance between the medical device 510 and the target anatomy 515. Such a sensor can include or be coupled to, for example, optical light, lasers, or ultrasonic sensors. The system can further include a position measuring device 520 positioned to make physical contact with the anatomy so that device moves with the anatomy to measure position. Ultrasound or other imaging modalities can also be used to observe deeper anatomical features. For example, it may be desirous to insert the tip of the probe to a specific cortical layer of the cerebral cortex.

Alternatively, other physiological measurements or monitor(s) 525 such as an ECG or heart rate monitor can track heart beats and the pulsing of anatomy can be anticipated based on this physiological data. Electrocardiogra respiratory rate, or other physiological measurement may be combined with the imaging modalities mentioned above to improve upon timing accuracy.

The system may also be configured to take electric measurements as a way of measuring or determining anatomical position. A sensor can be used to measure an electrical state of the anatomy. For example, the medical device 505 (such as the probe 105) can include multiple electrode tips with a corresponding electrode circuit. Each electrode's circuit is electrically isolated while the device is in air (or not contacting cortical surface.) The electrode circuits are not isolated while an electrical tip is contacting the cortical surface. Therefore, brain electrical activity or channel-to-channel resistance may be measured as a feedback to determine if the probe is contacting the brain. Using the above-mentioned imaging modalities and/or measurements, the thickness and/or durability of intact meningeal layer(s) may be determined. It should be appreciated that various systems and methods described herein can be configured for use with various anatomy and that the brain is a non-limiting example.

Any of a variety of mechanisms can be employed to achieve movement of the medical device. For example, the fixture 110 (or any medical device) may be moved by electrical motor, pneumatics or other methods or mechanisms. If a device is making contact with the anatomy then the device may be ‘floated’ above the anatomy so that it is physically raised/lowered by the force of the anatomy.

Any of a variety of mechanisms can be employed to achieve action such as inserting the probe during a specific anatomical state. For example, the probe may be inserted when an electrocardiogram (ECG) detects a heartbeat. Alternatively, the probe may be inserted when a sensor determines and provides feedback that the anatomy is ‘raised’ near the probe. The insertion may start automatically based on anatomy or it may require multiple triggers such as detecting a heartbeat and the operator manually actuating an actuator such as pressing a button.

The above-mentioned actions may be further modulated by information about the meningeal layer(s). For example, if it is desired to insert the probe through intact dura mater, the force required is likely higher than if it the insertion were to occur only through intact arachnoid and pial layers, and higher still if only the pia mater of the meninges is intact.

The example described herein shows an array probe with multiple electrodes being implanted in the cortical surface. However, the same methodology could be used for other devices such as single point probe, deep brain stimulation (DBS) electrodes, Stereoelectroencephalography (sEEG) depth electrodes, lasers, cannulas, needles, etc. The example described herein also shows the device making contact with the outer surface of the brain. The same methodology could also be used for targets that are not superficial (e.g. targets deeper in the anatomy such as specific neuronal layers or deep brain structures) or to hold devices proximate but not contacting the anatomy. The example described herein shows a device going into the brain. The same methodology could also be applied to other anatomical regions (e.g. spinal cord, blood vessels, heart, lungs, etc.) The example described herein shows a device being inserted through meningeal layers. The same methodology could be extended to other anatomical structures with varying superficial/anatomical layers (e.g., the fibrous capsule and renal cortex of the kidney).

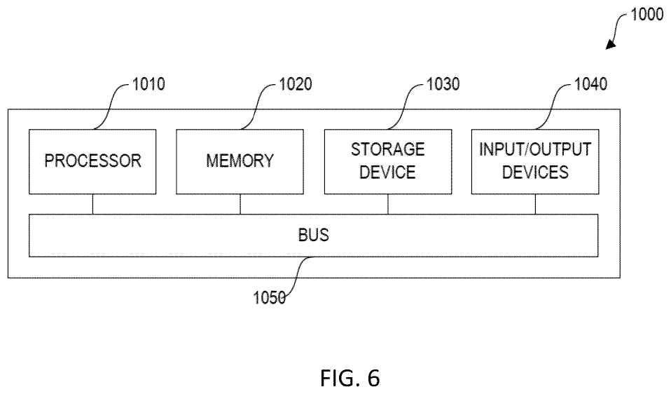

FIG. 6 depicts a block diagram illustrating an example of a computing control system consistent with implementations of the current subject matter. The computing control system 1000 may implement processes and methods described herein such as pursuant to control and movement of the medical device relative to the anatomy. As shown in FIG. 6, the computing system 1000 can include a processor 1010, a memory 1020, a storage device 1030, and input/output device 1040. The processor 1010, the memory 1020, the storage device 1030, and the input/output device 1040 can be interconnected via a system bus 1050. The processor 1010 is capable of processing instructions for execution within the computing system 1000. Such executed instructions can implement one or more components of, for example, VESA, 3D-ID AI, and/or MP Tool. In some implementations of the current subject matter, the processor 1010 can be a single-threaded processor. Alternately, the processor 1010 can be a multi-threaded processor. The processor 1010 is capable of processing instructions stored in the memory 1020 and/or on the storage device 1030 to display graphical information for a user interface provided via the input/output device 1040.

The memory 1020 is a computer readable medium, such as volatile or non-volatile, that stores information within the computing system 1000. The memory 1020 can store data structures representing configuration object databases, for example. The storage device 1030 is capable of providing persistent storage for the computing system 1000. The storage device 1030 can be a floppy disk device, a digital cloud, a hard disk device, an optical disk device, or a tape device, or other suitable persistent storage means. The input/output device 1040 provides input/output operations for the computing system 1000. In some implementations of the current subject matter, the input/output device 1040 includes a keyboard and/or pointing device. In various implementations, the input/output device 1040 includes a display unit for displaying graphical user interfaces. According to some implementations of the current subject matter, the input/output device 1040 can provide input/output operations for a network device. For example, the input/output device 1040 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 1000 can be used to execute various interactive computer software applications that can be used for organization, analysis and/or storage of data in various (e.g., tabular) format (e.g., Microsoft Excel®, and/or any other type of software). Alternatively, the computing system 1000 can be used to execute any type of software application. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug ins, or can be standalone computing products and/or functionalities. Upon activation within the applications, the functionalities can be used to generate the user interface provided via the input/output device 1040. The user interface can be generated and presented to a user by the computing system 1000 (e.g., on a computer screen monitor, etc.). The user interface can be integrated with other devices or virtual ecosystems.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

Any processes and/or logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A method of synchronizing a medical device intervention, comprising:
positioning a medical device relative to anatomy;
obtaining data regarding a position of the anatomy;
using the data to synchronize a position of the medical device relative to the anatomy.

2. The method of claim 1, wherein synchronizing a position of the medical device relative to the anatomy comprises moving the medical device in sync with the position of the anatomy.

3. The method of claim 1, wherein synchronizing a position of the medical device relative to the anatomy comprises performing a function on the anatomy while the anatomy is in a specific state.

4. The method of claim 3, wherein the specific state comprises a pulse state.

5. The method of claim 1, wherein the anatomy comprises a cortical surface.

6. The method of claim 1, wherein obtaining data regarding a position of the anatomy comprises taking an electrical measurement of the anatomy measure or determine an anatomical position of the anatomy.

7. The method of claim 1, wherein obtaining data regarding a position of the anatomy comprises using a sensor coupled to optical light data, laser data, or ultrasonic data.

8. The method of claim 6, wherein the medical device includes at least one electrode that provides feedback on whether the medical device is in contact with the anatomy.

9. The method of claim 2, wherein a distal end of the medical device is moved in sync with the position of the anatomy.

10. A medical device positioning system configured to synchronize a relative position between a medical device and anatomy, comprising:
a medical device including a probe;
at least one data processor; and
at least one memory including computer program code, the at least one memory and the computer program code configured to, with the at least one data processor, cause the system, to at least:
obtain medical device data regarding a position of medical device relative to anatomy;
obtaining anatomy data regarding a position of the anatomy;
using the medical device data and the anatomy data to synchronize a position of the medical device relative to the anatomy as the anatomy moves.

11. The system of claim 10, wherein the at least one memory and the computer program code are further configured to, with the at least one data processor, cause the system, to at least move the medical device in sync with the position of the anatomy.

12. The system of claim 10, wherein the at least one memory and the computer program code are further configured to, with the at least one data processor, cause the system, to perform a function on the anatomy while the anatomy is in a specific state.

13. The system of claim 12, wherein the specific state comprises a pulse state.

14. The system of claim 12, wherein the anatomy comprises a cortical surface.

15. The system of claim 10, wherein the at least one memory and the computer program code are further configured to, with the at least one data processor, cause the system, to take an electrical measurement of the anatomy measure an anatomical position of the anatomy relative to the medical device.

16. The system of claim 10, wherein the at least one memory and the computer program code are further configured to, with the at least one data processor, cause the system, to use a sensor coupled to optical light data, laser data, or ultrasonic data.
